(19) United States
(12) Patent Application Publication
(10) Pub. No.: US 2010/0021569 A1
(43) Pub. Date: Jan. 28, 2010

(54) Use of Extracts from AMTHS plants in Lowering Blood Glucose

(76) Inventor: Klim King, Taipei (TW)

Correspondence Address: G. LINK CO., LTD., 3550 BELL ROAD, MINOOKA, IL, 60447, US

(21) Appl. No.: 12/533,981

(22) Filed: Jul. 31, 2009

(30) Foreign Application Priority Data
Jul. 4, 2008 (CN) 200820109045.2

(51) Int. Cl. A61K 36/00; A61P 7/00

(52) U.S. Cl. 424/725

(57) ABSTRACT

The present invention discloses the use of extracts from the plants of the genus Amaranthus, such as the extracts from Amaranthus mangostanus L. and Amaranthus tricolor L., in lowering the blood glucose level. The present invention further provides anti-diabetes compositions or blood glucose-regulating dietary supplements, which contain the Amaranthus plant extracts as active agent.

### FIELD OF THE INVENTION

The present invention relates to the use of crude extracts or extracts from the plants of the genus Amaranthus in lowering blood glucose levels in mammals.

### BACKGROUND OF THE INVENTION

Many ethnobotanical surveys on medicinal plants used by the local population have been performed in different parts of the world. Several plant species have been described as hypoglycemic. Although phytotherapy continues to be used in several countries, few plants have received scientific or medical scrutiny. Moreover, a large number of medicinal plants possess some degree of toxicity. For example, it was reported that about one third of medicinal plants used in the treatment of diabetes is considered to be toxic.

AMTHS are plants of the genus Amaranthus. There are approximately 60 species; all are annuals with small seeds (approximately 0.07 grams per 100 seeds). The cultivated forms are useful for producing nutritious grain and foliage and as colorful ornamentals. Amaranthus mangostanus L. and Amaranthus tricolor L. are two common dietary vegetables that are easily available from local Taiwan markets. In the present invention, it was unexpectedly found that crude extracts or extracts of Amaranthus mangostanus L. and Amaranthus tricolor L. effectively lower postprandial glucose excursion.

In the present invention, glucose tolerance tests were performed to determine whether extracts from Amaranthus mangostanus L. and Amaranthus tricolor L. lower the postprandial blood glucose level in rodents. Experimental results of present invention revealed that the water extract from Amaranthus tricolor L. and the ethyl acetate extract from Amaranthus mangostanus L. significantly decreased postprandial blood glucose levels in mice and that these plants may be developed into medicines or dietary supplements to regulate blood glucose levels.

### SUMMARY OF THE INVENTION

The present invention is based on the unexpected discovery that the extract from plants in genus Amaranthus, such as Amaranthus mangostanus L. and Amaranthus tricolor L., can lower the postprandial blood glucose level in rodents.

Therefore, the invention provides a pharmaceutical composition comprising the extract from plants in genus Amaranthus as an active agent

The “mammal” mentioned herein may be a rodent, such as mouse, rat, rabbit, or may be a human. The human may be a diabetic patient or a person with hyperglycemia.

In one embodiment of the invention, the extract is the water extract from Amaranthus tricolor L. In another embodiment of the invention, the extract is the ethyl acetate extract from Amaranthus mangostanus L. In a further embodiment of the invention, the pharmaceutical composition is used for treating or preventing a disease that results from an abnormality in serum glucose, such as diabetes.

In another aspect, the invention provides a dietary supplement for the regulation of blood glucose in which a processed product derived from the genus Amaranthus acts as the primary ingredient. The dietary supplement may be formulated as either a food, beverage or feed, which can be used in treating or preventing a disease that results from an abnormality in human serum glucose.

In an additional embodiment of the invention, the processed product used in the blood glucose-regulating dietary supplement is the water extract from Amaranthus tricolor L. In another embodiment of the invention, the processed product is the ethyl acetate extract from Amaranthus mangostanus L.

The details regarding one or more embodiments of the invention are set forth in the description below. Other features or advantages of the present invention will be apparent from the following drawings and detailed description of one example, and also from the appended claims.

### BRIEF DESCRIPTION OF THE DRAWINGS

The drawings are first described.

FIG. 1 is a diagram showing the effect of the extract from Amaranthus tricolor L. on the postprandial glucose levels after an I.P. glucose tolerance test in lean C57BL/6B mice. Amaranthus tricolors L. extract () or vehicle (⋄) was orally administrated to mice followed by an I.P. glucose challenge (10 g/kg).

FIG. 2 is a diagram showing the effect of Amaranthus mangostanus L. on postprandial glucose levels after an I.P. glucose tolerance test in lean C57BL/6N mice. Amaranthus mangostanus L. extract () or vehicle (⋄) was orally administrated to mice followed by an I.P. glucose challenge (10 g/kg).

### DETAILED DESCRIPTION OF THE INVENTION

Described herein is the hypoglycemic effect of extracts from Amaranthus plants, such as Amaranthus mangostanus L. and Amaranthus tricolor L., on mouse postprandial glucose excursion.

Based on the hypoglycemic effect, the present invention provides a pharmaceutical composition for lowering serum level of glucose, which comprises an extract from a plant of genus Amaranthus as its active agent. The pharmaceutical compositions of the present invention may be prepared by conventional techniques, e.g. as described in Remington's Pharmaceutical Sciences, 1985 or in Remington: The Science and Practice of Pharmacy, 19^{th} edition, 1995.

In one embodiment the pharmaceutical formulation is a freeze-dried formulation, whereto the physician or the patient adds solvents and/or diluents prior to use. In another embodiment the pharmaceutical formulation is a dried formulation (e.g. freeze-dried or spray-dried) ready for use without any prior dissolution.

Thus, the injectable compositions of the Amaranthus plant extract of the invention can be prepared using the conventional techniques of the pharmaceutical industry which involves dissolving and mixing the ingredients as appropriate to give the desired end product.

The Amaranthus plant extracts of the invention may be used for the preparation of a medicament for the treatment or prevention of hyperglycemia, type 2 diabetes, impaired glucose tolerance, type 1 diabetes, obesity, hypertension, decreasing food intake, decreasing β-cell apoptosis, increasing β-cell function and β-cell mass, restoring glucose sensitivity to β-cells, and/or for delaying or preventing disease progression in type 2 diabetes.

Administration of pharmaceutical compositions according to the invention may be through several routes of administration (such as lingual, sublingual, buccal, in the mouth, oral, in the stomach and intestine, epidermal, dermal, transdermal, and parenteral) to patients in need of such a treatment.

Compositions of the present invention may be administered in several dosage forms, such as solutions, suspensions, emulsions, microemulsions, multiple emulsion, foams, salves, pastes, plasters, ointments, tablets, coated tablets, capsules, hard gelatine capsules and soft gelatine capsules, drops, gels, sprays, powder, injection solutions, in situ transforming solutions, in situ gelling, in situ setting, in situ precipitating, in situ crystallization, infusion solution, and implants.

The treatment with a plant extract according to the present invention may also be combined with a second, more pharmacologically active substances and may be selected from antidiabetic agents, antiobesity agents, appetite regulating agents, antihypertensive agents, agents for the treatment and/or prevention of complications resulting from or associated with diabetes and agents for the treatment and/or prevention of complications and disorders resulting from or associated with obesity. In the present context the expression “antidiabetic agent” includes compounds for the treatment and/or prophylaxis of insulin resistance and diseases wherein insulin resistance is the pathophysiological mechanism. It should be understood that any suitable combination of the compounds according to the invention with one or more of the above-mentioned compounds and optionally one or more further pharmacologically active substances are considered to be within the scope of the present invention.

The Amaranthus plant extracts of the invention may be used for the preparation of a dietary supplement for the regulating blood glucose. The dietary supplement of the invention may be prepared in the form of solids, semi-solids, gels, syrups, suspensions or solutions. The dietary supplements may also contain buffer salts, flavoring, coloring and sweetening agents as appropriate.

Without further elaboration, it is believed that one skilled in the art can, based on the above description, utilize the present invention to its fullest extent.

The following specific example is therefore to be construed as merely illustrative and not limitative of the remainder of the disclosure in any way whatsoever. All publications cited herein are incorporated by reference.

### Preparation of Plant Extracts

Fresh Amaranthus mangostanus L. and fresh Amaranthus tricolor L. were bought from the local whole sale market in Taipei city from April to September 2008. The fresh plants were dried at 60° C. in an oven incubator for 8 hours and then crushed into 20 mesh particles with a size gated blender. To prepare the water extract, the powder of Amaranthus tricolor L. was extracted with distilled water (100 g/1000 ml) and constantly stirred at 90° C. for 2 h, and then filtered through a filter paper. Residue was again extracted as above with water at 90° C. for 2 h. The combined filtrate was evaporated with a rotary evaporator at 50° C. under reduced pressure and freeze-dried in a lyophilizer. The yield of the Amaranthus tricolor L. water extract was approximately 15% of the plant powder.

The ethyl acetate extract of plant Amaranthus mangostanus L. powder was prepared by extracting 100 grams of Amaranthus mangostanus L. powder in 1000 ml of ethyl acetate at 25° C. combined with constant stirring for 16 h, and then filtered through a filter paper. The filtrate from the extraction was first evaporated with a rotary evaporator at 40° C. under reduced pressure and followed by drying in a lyophilizer. The yield of Amaranthus mangostanus L. ethyl acetate extract was estimated to be 8% of the dried plant material.

To study the hypoglycemic effect of extracts, extract solutions were freshly prepared by dissolving dried plant extracts in 10% Tween 20 to give a final concentration of 0.02 gram per ml. To facilitate the dissolving process, the solution was incubated in a sonication bath at 60° C. for 30 min.

### Glucose Tolerance Test

### Method:

Six-weeks old C57B16 mice with a range of body weight between 20 to 25 g were purchased from Charles River, Animal Center, Taiwan, ROC and used for the study. All mice were housed 6 per polycarbonate cage and fed with feed with Laboratory Rodent Diet 5001 (PMI Nutrition International, Inc., MO, USA) in the AAALAC accredited animal facility. The temperature was maintained at 21° C. with humidity kept between 50% to 70%. The light cycles were 12 h light and 12 h dark and individual mouse was identified by ear notch. Before each experiment, the animals were fasted for 16 h. The experimental protocols were approved by the Institutional Animal Ethical Committee.

Mice were fasted overnight (16 h), then fed with Laboratory Rodent Diet 5001 and supplied with water ad libitum for 2 h followed by fasting for 1 h. At the end of 1 h fasting, mice were dosed orally 10 ml/kg of the extract solution in 10% Tween 20 containing 0.04 g of extract per ml or were dosed with vehicle solution (10% Tween 20). The hypoglycemic effect was assessed by intraperitoneal glucose tests 1 h after administering the extract solution. Blood samples were drawn from the tail vein at 0, 30, 60, and 90 min after glucose administration. Blood glucose levels were measured using a Glucometer.

### Result:

To investigate the effect of Amaranthus tricolor L. extract on postprandial plasma glucose excursion, mice were fed for 2 h followed by 1 h fasting then dosed with extracts or vehicle. The effect of Amaranthus tricolor L. extract on the postprandial regulation of blood glucose was examined by monitoring the glucose increment in mice after an exogenous i.p. glucose administration. A postdose, preglucose sample was obtained at 0 min, and then an IP glucose load was administered (10% glucose; 10 ml/kg). Postload blood samples were obtained at 30, 60, and 90 min.

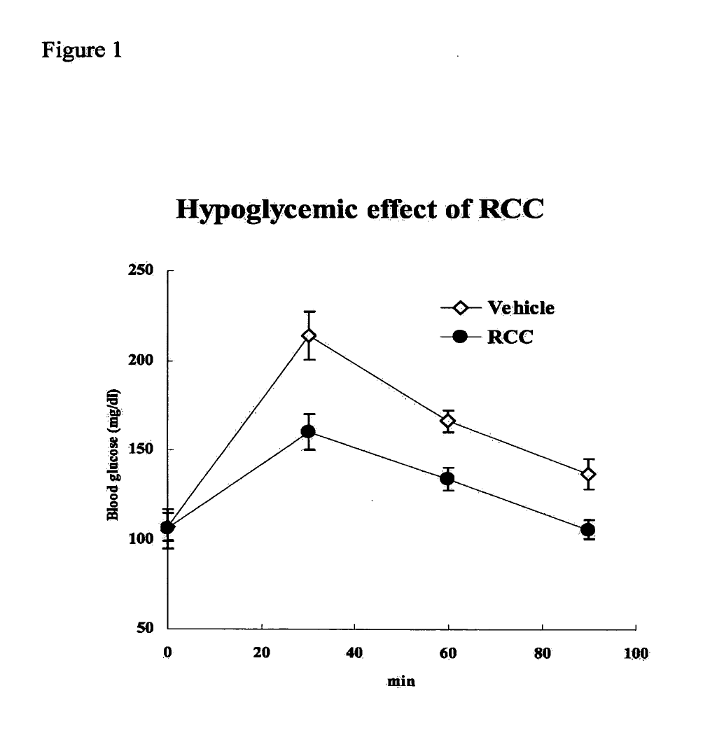

As shown in FIG. 1, plasma glucose increased from 105 mg/ml to more than 200 mg/ml 30 min after a mouse received an i.p. glucose challenge, and the level of plasma glucose gradually returned to 140 mg/ml 90 min after glucose administration. In mice dosed with 50 mg/kg water extract of Amaranthus tricolor L., the blood glucose level increased to 158 mg/ml 30 min after glucose challenge and returned to 107 mg/ml 90 min after glucose administration. Baseline plasma glucose levels were nearly identical in mice assigned the water extract from Amaranthus tricolor L. (105 mg/ml) and vehicle (104 mg/ml) and did not change appreciably until the exogenous glucose load was administered. Both the mean peak glucose (158 mg/ml) and the incremental area under the curve (AUC) 0-90 min were significantly lower in mice receiving Amaranthus tricolor L. extract than in vehicle-treated mice (215 mg/ml).

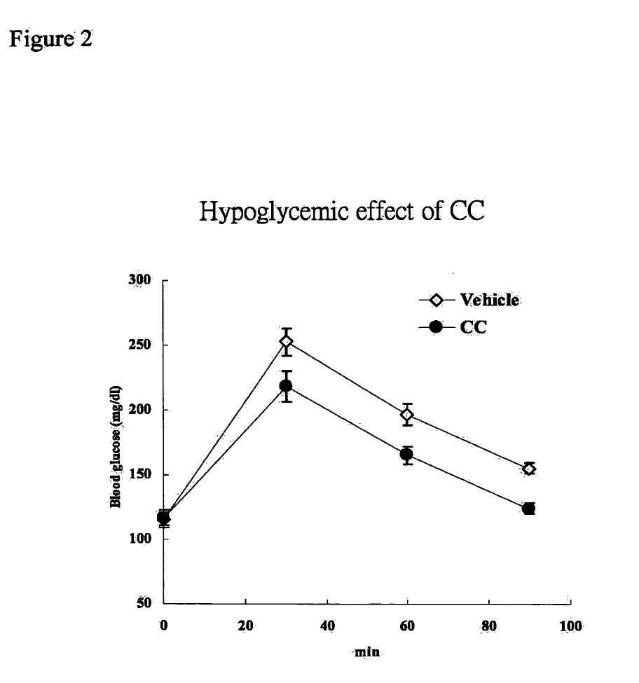

A similar effect was also found in ethyl acetate extract from Amaranthus mangostanus L. as shown in FIG. 2. Although the baseline plasma glucose levels were similar, the mean peak glucose (220 mg/ml) in mice received Amaranthus mangostanus L. extract was lower than that (270 mg/ml) of vehicle treated mice, and the incremental area under the curve (AUC) 0-90 min was significantly lower in mice receiving Amaranthus mangostanus L. extract than in vehicle-treated mice.

The results described above demonstrate that a single dose of extract from Amaranthus tricolor L. or Amaranthus mangostanus L. significantly reduces postprandial plasma glucose level. The hypoglycemic activity found in these two plants of genus Amaranthus is worth noting. Since both Amaranthus tricolor L. and Amaranthus mangostanus L. are common vegetables routinely consumed in Taiwan and China for years, it is relative safe to use these extracts to control plasma glucose. Therefore, the extracts from Amaranthus plants are potential active ingredients for developing anti-diabetes drugs and blood glucose-regulating dietary supplements.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention and without departing from the spirit and scope thereof, they can make various changes and modifications to the invention to adapt it to various usages and conditions. Thus, other embodiments are also within the claims.

What is claimed is:

1. A pharmaceutical composition for lowering serum level of glucose in mammal, which comprises an extract from a plant of genus Amaranthus as active agent and a pharmaceutically acceptable vehicle.

2. The pharmaceutical composition of claim 1, wherein the mammal is a human.

3. The pharmaceutical composition of claim 2, wherein the human is a diabetic patient with hyperglycemia.

4. The pharmaceutical composition of claim 1, wherein the extract is derived from Amaranthus mangostanus L.

5. The pharmaceutical composition of claim 4, wherein the extract is obtained from the extraction of Amaranthus mangostanus L. with ethyl acetate at 20-30° C.

6. The pharmaceutical composition of claim 1, wherein the extract is derived from Amaranthus tricolor L.

7. The pharmaceutical composition of claim 6, wherein the extract is obtained from the extraction of Amaranthus tricolor L. with water at 80-95° C.

8. The pharmaceutical composition of claim 4, wherein the extract is lyophilized.

9. A dietary supplement for the regulating blood glucose, which is characterized in that the dietary supplement comprises an extract from a plant of genus Amaranthus as the effective ingredient.

10. The dietary supplement of claim 9, wherein the dietary supplement is formulated in a form of food, beverage or feed.

11. The dietary supplement of claim 9, wherein the processed product is the water extract from Amaranthus tricolor L.

12. The dietary supplement of claim 9, wherein the processed product is the ethyl acetate extract from Amaranthus mangostanus L.

13. A method of glycemic control in mammal, which comprises administering an effective amount of pharmaceutical composition of claim 1 to a mammal in need thereof.

14. The method of claim 13, wherein the mammal is a human with hyperglycemia.

15. The method of claim 14, wherein the human is a diabetic patient.

16. The pharmaceutical composition of claim 6 wherein the extract is lyophilized.
